(19) United States
(12) Patent Application Publication
(10) Pub. No.: US 2025/0120980 A1
(43) Pub. Date: Apr. 17, 2025

(54) DIBENZODIAZEPINE COMPOUNDS AND USE IN INTERFERON ACTIVATOR PROTEIN AGONISTS THEREOF

(71) Applicant: HEBEI GRANDIOS PHARMA CO, LTD., Hengshui (CN)

(72) Inventors: Shangjin YANG, Hengshui (CN); Haijun LV, Hengshui (CN); Jiajia GUO, Hengshui (CN); Meng CHEN, Hengshui (CN); Xiaoli WANG, Hengshui (CN); Yingli SHANG, Hengshui (CN); Meng WANG, Hengshui (CN)

(73) Assignee: HEBEI GRANDIOS PHARMA CO, LTD., Hengshui (CN)

(21) Appl. No.: 18/813,075

(22) Filed: Aug. 23, 2024

(30) Foreign Application Priority Data
Aug. 29, 2023 (CN) 202311097987.9

(51) Int. Cl. A61K 31/55; A61P 31/12

(52) U.S. Cl. CPC A61K 31/55; A61P 31/12

(57) ABSTRACT

The present invention discloses benzodiazepine compounds and use in interferon activator protein agonists thereof, involving a compound of formula I, a racemate or optical isomer thereof, a pharmaceutically acceptable salt and solvate thereof, and use in the preparation of antiviral and anti-tumor drugs related to interferon activator protein (STING) thereof.

### TECHNICAL FIELD

The present invention belongs to the field of medicine, and specifically relates to dibenzodiazepine compounds and use in interferon activator protein agonists thereof.

### BACKGROUND

Cancer is a general term for a large type of malignant tumor, which is currently a major stubborn disease that seriously endangers human life and health. According to data from the National Cancer Center Research Institute [Journal of National Cancer Center, 2022(2): 1-9], 2016, there were approximately 4.06 million new cancer cases and 2.41 million deaths in China. Among them, lung cancer, colon cancer, rectal cancer, stomach cancer, liver cancer and breast cancer account for 57.4% of the total number of new cancer cases. Lung cancer is the most common cancer in men, accounting for 24.6% of all new cancer cases. Breast cancer is the most common cancer case among women, accounting for 68.83% of all new cancer cases. In recent years, due to the impact of objective factors such as lifestyle change, population aging, environmental pollution, etc., the incidence rate of malignant tumors in China has continued to rise, becoming the first deadly disease. Globally, China accounted for 23.7% of new cancer cases in 2020, and it is expected that the cancer burden will increase by 50% compared to 2020 in 2040. Faced with the increasingly severe situation of anti-cancer, the solution to the problem of anti-cancer is urgent.

Cancer is a disease caused by excessive proliferation of cells in the body after mutation, which disrupts the normal functioning of surrounding tissues, and known as malignant tumors. The main genetic changes include the transformation of proto-oncogenes into oncogenes and the inactivation of tumor suppressor genes. The clinical manifestations of malignant tumors vary depending on their location and degree of development, but early malignant tumors often have no obvious symptoms. By the time specific symptoms appear in patients, the tumor has already entered the late stage. Due to the diversity of tumors, the specificity of their distribution range, and the differences in human physical fitness, most patients require comprehensive diagnosis and treatment. Methods such as surgery, chemotherapy, physical radiation therapy, immunotherapy, and traditional Chinese medicine treatment are commonly used. Among them, drug therapy is an important link. In recent decades, with the advancement of life science technology, drug therapy has made significant progress. It has gradually developed from traditional cytotoxic drugs to molecular targeted drugs, immunotherapy, and biological agents. The treatment efficacy of patients has been significantly improved, and the survival period has been extended. However, most traditional anti-cancer drugs have shortcomings such as low safety, multiple side effects, and slow onset. Therefore, promoting the development of innovative anti-cancer drugs is of great practical significance in the field of anti-tumor treatment.

With the continuous development of tumor molecular biology and genomics, molecular targeted therapy and immunotherapy have become novel approaches for current tumor treatment. Immunotherapy has especially become one of the current research hotspots, as the innate immune response is the host's first line of defense against foreign pathogenic microorganisms. The natural immune system recognizes foreign pathogens through pattern recognition receptors, activates signal transduction pathways, induces the secretion of interferons, inflammatory factors, and other factors, thereby enabling host cells to develop defense capabilities. Interferon activator protein (STING) is an important molecule in innate immune response, mainly exerting immune defense and anti-tumor effects through the cGAS-STING pathway. With the report of STING crystal structure, it provides a certain foundation for the research of discovering new STING agonists through structure-based drug design. Therefore, STING is a very meaningful drug target that can activate the STING pathway by designing STING agonists, thereby exerting anti-tumor, antiviral, and antibacterial effects. The research on STING agonists has also received widespread attention from the scientific community. STING (interferon activator protein) agonists have been one of the focuses of molecular biology research in recent years, and have been widely used as immunotherapy targets in clinical studies. Experimental results have shown that it is closely related to research on virus proliferation, gene transcription, and anticancer drugs. Many drugs, such as NBTXR-3, exoSTING, IMSA-101, etc., exert inherent immune effects of cells by affecting the STING pathway. Therefore, STING is often used as a new target for screening anti-cancer drugs in immunotherapy (STING, a promising target for small molecular immune modulator: A review, European Journal of Medicinal Chemistry 211 (2021) 113113; The cGAS-STING Defense Pathway and Its Counteraction by Viruses, Cell host & microbe, 2016, 19(2): 150-158). Therefore, many compounds have already entered clinical practice (The Potential of STING Agonists Is Explored in Cancer, Targeted Therapies in Oncology, 2022, 11(4), 63). Although there are many drugs available as interferon activator protein agonists, their chemical structures are often complex, synthesis is difficult, and some are even biological products, making them expensive. Therefore, further research is needed on anti-cancer related drugs.

### SUMMARY OF THE INVENTION

The present invention relates to dibenzodiazepine compounds and use in interferon activator protein agonists thereof, obtaining small molecule compounds with simple chemical structures and concise synthesis as interferon activator protein agonists will help solve existing problems.

We are delighted to discover, through chemical synthesis and biological experiments, that dibenzo[b,f]diazepine within the range of chemical structural formulas has the activity of stimulating interferon activator protein (STING), and can therefore be used as antibacterial, antiviral, and anti-tumor drugs.

The technical solution of the present invention is as follows:

A dibenzodiazepine compound, the dibenzodiazepine compound is a compound of formula I or a stereoisomer thereof, or a pharmaceutically acceptable salt or solvate thereof:

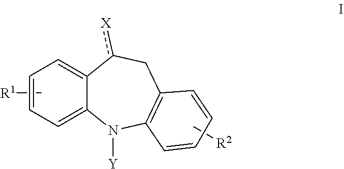

wherein,
X is hydrogen, oxygen, sulfur, C1-6 alkoxy, and C1-6 alkylmercapto;
Y is hydrogen, alkyl, carbamoyl, N—C1-6 alkyl-substituted carbamoyl, acetyl, or other acyls containing 3-10 carbons;
wherein, R1 and R2 are respectively 0-5 different substituents optionally existing on their respective benzene rings, and are independently selected from the following groups: hydrogen, halogen, C1-6 alkyl, hydroxyl, C1-6 haloalkyl, C1-6 alkoxy, amino, amino mono-or di-substituted with C1-6 alkyl, carboxyl, acetate, amide, and phenyl.

Preferably, the dibenzodiazepine compound is a compound of formula II or a stereoisomer thereof, or a pharmaceutically acceptable salt or solvate thereof:

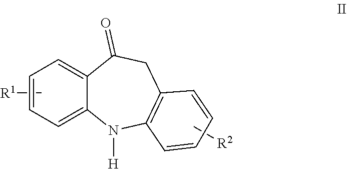

wherein, R1 and R2 are respectively 0-5 different substituents optionally existing on their respective benzene rings, and are each independently selected from the following groups: hydrogen, halogen, C1-6 alkyl, hydroxyl, C1-6 haloalkyl, C1-6 alkoxy, amino, amino mono-or di-substituted with C1-6 alkyl, carboxyl, acetate, amide, and phenyl.

Further preferably, it is a compound of formula II, wherein R1 is optionally 1-3 different substituents, and is each independently selected from the following groups: hydrogen, halogen, C1-6 alkyl, hydroxyl, and C1-6 alkoxy.

Further preferably, it is a compound of formula II, wherein R2 is optionally 1-3 different substituents, and is each independently selected from the following groups: hydrogen, and C1-6 alkoxy.

The dibenzodiazepine compound according to any one above, the dibenzodiazepine compound is selected from the following compounds:
5,11-dihydro-10H-dibenzo[b,f]aza-10-one,
or 6,7-dimethyl-5,11-dihydro-10H-dibenzo[b,f]aza-10-one,
or 6,7-dimethyl-5,11-dihydro-10H-dibenzo[b,f]aza-10-ol,
or 7,8-dimethyl-5,11-dihydro-10H-dibenzo[b,f]aza-10-one,
or 6-chloro-5,11-dihydro-10H-dibenzo[b,f]aza-10-one,
or 7-chloro-5,11-dihydro-10H-dibenzo[b,f]aza-10-one,
or 8-methoxy-5,11-dihydro-10H-dibenzo[b,f]aza-10-one,
or 3-methoxy-6,7-dimethyl-5,11-dihydro-10H-dibenzo[b,f]aza-10-one,
or 2-methoxy-6,7-dimethyl-5,11-dihydro-10H-dibenzo[b,f]aza-10-one,
or 3-fluoro-6,7-dimethyl-5,11-dihydro-10H-dibenzo[b,f]aza-10-one,
or 8-hydroxy-5,11-dihydro-10H-dibenzo[b,f]aza-10-one.

Further preferably, a synthesis intermediate of the dibenzodiazepine compound is a compound of formula III:
wherein, R¹ and R² are respectively 0-5 different substituents optionally existing on

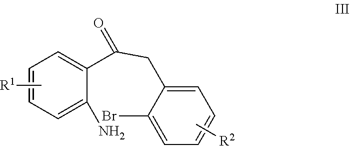

their respective benzene rings, and are each independently selected from the following groups: hydrogen, halogen, C₁₋₆ alkyl, hydroxyl, C₁₋₆ haloalkyl, C₁₋₆ alkoxy, amino, amino mono- or di-substituted with C₁₋₆ alkyl, carboxyl, acetate, amide, and phenyl.

Further preferably, the compound of formula III is obtained by reacting substituted aniline and substituted o-bromobenzonitrile as raw materials in the presence of boron trichloride and aluminum trichloride, with the reaction pathway as follows:

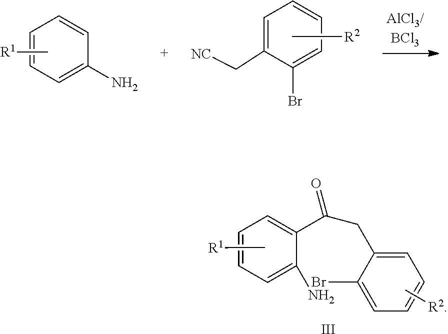

The benzodiazepine compound or a stereoisomer thereof, or a pharmaceutically acceptable salt or solvate thereof in the preparation of drugs for the treatment or prevention of tumors related to the STING pathway, as well as diseases caused by viruses and bacteria.

The following Table I lists the compound numbers, chemical names, and chemical structures of a compound of formula II, which are representative of compounds of formulas I and II. These compounds were prepared by the intramolecular amination reaction of bromobenzene using the intermediate mentioned above:

TABLE I

| Compound | | Structural |
|---|---|---|
| No. | Name | formula |
| 11 | 6,7-dimethyl-5,11-dihydro-10H- dibenzo[b,f]aza-10-one | 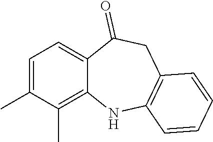 [SMILES: Cc1ccc2c(c1C)Nc1ccccc1CC2=O] |
| 11a | 6,7-dimethyl-5,11-dihydro-10H- dibenzo[b,f]aza-10-ol | 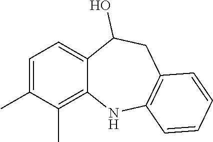 [SMILES: Cc1ccc2c(c1C)Nc1ccccc1CC2O] |
| 12 | 7,8-dimethyl-5,11-dihydro-10H- dibenzo[b,f]aza-10-one | 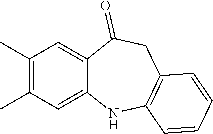 [SMILES: Cc1cc2c(cc1C)C(=O)Cc1ccccc1N2] |
| 13 | 3-fluoro-6,7-dimethyl-5,11-dihydro-10H- dibenzo[b,f]aza-10-one | 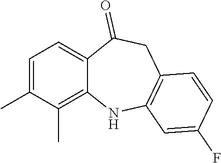 [SMILES: Cc1ccc2c(c1C)Nc1cc(F)ccc1CC2=O] |
| 14 | 5,11-dihydro-10H-dibenzodiazepine-10-one | 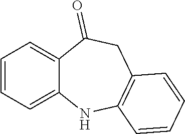 [SMILES: O=C1Cc2ccccc2Nc2ccccc21] |
| 15 | 6-chloro-5,11-dihydro-10H-dibenzo[b,f]aza- 10-one | 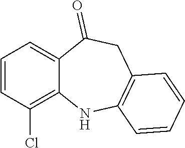 [SMILES: O=C1Cc2ccccc2Nc2c(Cl)cccc21] |
| 16 | 7-chloro-5,11-dihydro-10H-dibenzo[b,f]aza- 10-one | 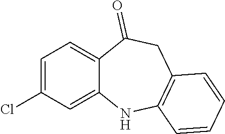 [SMILES: O=C1Cc2ccccc2Nc2cc(Cl)ccc21] |
| 17 | 8-methoxy-5,11-dihydro-10H-dibenzo[b,f] aza-10-one | 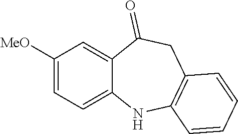 [SMILES: COc1ccc2c(c1)C(=O)Cc1ccccc1N2] |
| 18 | 3-methoxy-6,7-dimethyl-5,11-dihydro-10H- dibenzo[b,f]aza-10-one | 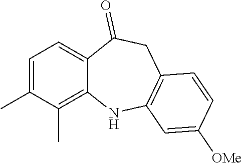 [SMILES: COc1ccc2c(c1)Nc1c(ccc(C)c1C)C(=O)C2] |
| 19 | 2-methoxy-6,7-dimethyl-5,11-dihydro-10H- dibenzo[b,f]aza-10-one | 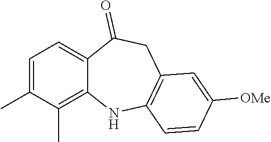 [SMILES: COc1ccc2c(c1)CC(=O)c1ccc(C)c(C)c1N2] |
| 20 | 8-hydroxy-5,11-dihydro-10H- dibenzo[b,f]aza-10-one | 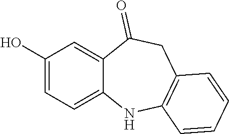 [SMILES: O=C1Cc2ccccc2Nc2ccc(O)cc21] |

The compounds of formulas I and II shown in the table have been tested for their biological activity and have shown significant induction of IFNb gene expression. Therefore, they can be used as antiviral and anti-tumor drugs related to interferon activator protein (STING).

Therefore, the present invention also provides compounds of formulas I and II or pharmaceutically acceptable salts thereof for anti-tumor and/or immunostimulatory properties.

The present invention further provides a pharmaceutical composition having anti-tumor and/or immunostimulatory properties and comprising at least one compound of formula I and II or a pharmaceutically acceptable salt thereof, and one or more pharmaceutically acceptable carriers or diluents.

Active compounds can be administered orally, such as with inert diluents or absorbable edible carriers, or they can be encapsulated in hard or soft shell gelatin capsules, or they can be compressed into tablets, or they can be consumed directly with food. For oral therapeutic administration, active compounds can be combined with excipients and used in the form of ingestible tablets, buccal tablets, troches, capsules, elixirs, suspensions, syrups, wafers, etc. Such compositions and formulations should contain at least 0.1% of active compounds. The percentage of the composition and formulation can of course vary and can conveniently be between about 2% and about 60% of the unit weight. The amount of active compound in the composition useful in such treatments will result in an appropriate dosage. A preferred composition or formulation according to the present invention was prepared such that the oral dosage unit form contained about 5 to about 200 milligrams of the active compound.

Tablets, troches, pills, capsules, etc. may also contain the following substances: binders, such as astragalus gum, arabic gum, corn starch, or gelatin; excipients such as dicalcium phosphate; disintegrating agents, such as corn starch, potato starch, alginate, etc; lubricants such as magnesium stearate; sweeteners such as sucrose, lactose, saccharin, or seasonings such as mint, holly oil, cherries can also be added. When the dosage unit form is a capsule, in addition to the above-mentioned types of materials, it can also contain a liquid carrier. Various other materials can exist as coatings or alter the physical form of dosage units in other ways. For example, tablets, pills, or capsules can be coated with shellac, sugar, or both. Syrups or elixirs may contain active compounds, sucrose as a sweetener, methyl p-hydroxybenzoate and ppropyl p-hydroxybenzoate as preservatives, dyes, and seasonings such as cherry or orange spices. Of course, any material used to prepare any dosage unit form should be pharmaceutically pure and essentially non-toxic in the amount used. In addition, active compounds can be incorporated into sustained-release formulations and formulations.

Active compounds can also be administered parenterally or intraperitoneally. Solutions of active compounds as free bases or pharmaceutically acceptable salts can be prepared by mixing them appropriately with surfactants such as hydroxypropyl cellulose in water. Dispersions can also be prepared in glycerol, liquid polyethylene glycol and their mixtures, and oil. Under normal storage and usage conditions, these formulations contain preservatives to prevent the growth of microorganisms.

The forms of drugs suitable for injection include sterile aqueous solutions or dispersions and sterile powders used for temporary preparation of sterile injectable solutions or dispersions. In all cases, such dosage forms must be sterile and fluid to the extent that they are easy to inject. It must be stable under manufacturing and storage conditions, and must prevent contamination by microorganisms such as bacteria and fungi. The carrier can be a solvent or dispersion medium, such as water, ethanol, polyols (such as glycerol, propylene glycol, and liquid polyethylene glycol) and their suitable mixtures, and vegetable oils. Appropriate fluidity can be maintained, for example, by using coatings such as phospholipids, by maintaining the desired particle size in dispersion, and by using surfactants. Various antibacterial and antifungal agents can be used to prevent the action of microorganisms, such as parabens, chlorobutanol, phenol, sorbic acid, merthiolate, etc. In many cases, isotonic agents such as sugar or sodium chloride are preferred. By using delayed absorbents such as aluminum monostearate and gelatin in the composition, the absorption of injectable compositions can be prolonged.

A sterile injectable solution was prepared by mixing the desired amount of active compounds with various other ingredients listed above into an appropriate solvent as needed, and then filtering and sterilizing. Dispersions are usually prepared by incorporating various sterilized active ingredients into sterile carriers, which contain a basic dispersion medium and other desired ingredients from those listed above. The preferred preparation methods for sterile powders used in the preparation of sterile injectable solutions are vacuum drying and freeze-drying techniques, which produce powders of active ingredients along with any additional desired ingredients from their previously sterile filtered solutions.

As used herein, “pharmaceutically acceptable carriers” include any and all solvents, dispersion media, coatings, antibacterial and antifungal agents, isotonic agents, and absorption retarders. The use of such media and reagents for drug active substances is well known in the art. Unless any conventional medium or reagent is incompatible with the active ingredient, its use in therapeutic compositions is expected. Supplementing active ingredients can also be incorporated into the composition.

It is particularly advantageous to prepare parenteral compositions in dosage unit form for easy administration and uniform dosage. The dosage unit form used herein refers to physically discrete units that are suitable as unit doses for mammalian subjects to be treated; Each unit contains a predetermined amount of active substance, which is calculated to bind with the desired drug carrier to produce the desired therapeutic effect. The specifications of the new dosage unit form of the present invention are determined by the following factors and directly depend on (a) the unique characteristics of the active substance and the specific therapeutic effect to be achieved, as well as (b) the limitations inherent in the field of mixing such drugs. An active substance is used to treat diseases in living subjects with impaired physical health.

The main active ingredient is mixed in an effective amount with a suitable pharmaceutically acceptable carrier in the dosage unit form as disclosed above for convenient and effective administration. For example, the unit dosage form may contain about 0.1 mg to about 400 mg of the main active compound, preferably about 1 mg to about 30 mg. Active compounds are typically present in a carrier at a concentration of about 0.1 mg/ml to about 400 mg/ml in proportion. In the case of a composition containing a supplementary active ingredient, the dosage is determined by referring to the commonly used dosage and administration method of the ingredient. The beneficial effects of the present invention are as follows:

The present invention uses benzodiazepine compounds as interferon activator protein agonists. The structure of the benzodiazepine compounds is simple and the synthetic route we have developed is concise. Therefore, once used as drugs, the price will be very favorable for patients.

The synthetic route of the present invention uses substituted aniline and substituted o-bromobenzonitrile as raw materials, which react in the presence of boron trichloride and aluminum trichloride, and then undergoes intramolecular Ullmann reaction or Buchwald reaction for cyclization to obtain substituted benzodiazepine. A total of at least two chemical reactions are required, which is shorter than traditional synthetic routes, more environmentally friendly, and cost-effective.

### DETAILED DESCRIPTION OF THE PREFERRED EMBODIMENTS

The present invention will be further elaborated with specific embodiments. However, the scope of protection claimed by the present invention is not limited to the scope described in the embodiments.

Table II lists the physical data of intermediate formula III for a compound of formula II

TABLE II

| Num- | Compound | Prop- | Melting |
|---|---|---|---|
| ber | structure | erty | point |
| 1 | 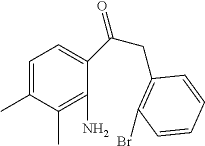 [SMILES: Cc1ccc(C(=O)Cc2ccccc2Br)c(N)c1C] | Green solid | 116-117° C. |
| 2 | 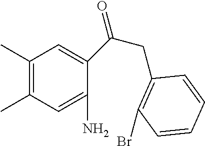 [SMILES: Cc1cc(N)c(C(=O)Cc2ccccc2Br)cc1C] | Orange solid | 108-109° C. |
| 3 | 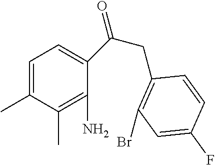 [SMILES: Cc1ccc(C(=O)Cc2ccc(F)cc2Br)c(N)c1C] | White crystal | 112-114° C. |
| 4 | 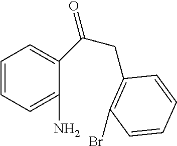 [SMILES: Nc1ccccc1C(=O)Cc1ccccc1Br] | Yellow solid | 88-90° C. |
| 5 | 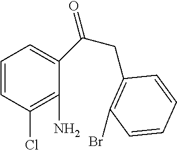 [SMILES: Nc1c(Cl)cccc1C(=O)Cc1ccccc1Br] | Cyan solid | 133-135° C. |
| 6 | 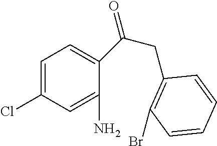 [SMILES: Nc1cc(Cl)ccc1C(=O)Cc1ccccc1Br] | Green solid | 115-117° C. |
| 7 | 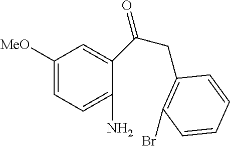 [SMILES: COc1ccc(N)c(C(=O)Cc2ccccc2Br)c1] | Green solid | 79-80° C. |
| 8 | 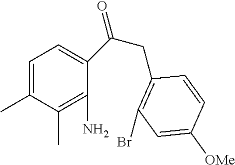 [SMILES: COc1ccc(CC(=O)c2ccc(C)c(C)c2N)c(Br)c1] | Yellow solid | 165-167° C. |
| 9 | 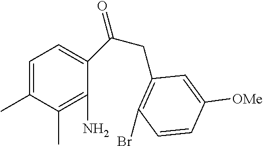 [SMILES: COc1ccc(Br)c(CC(=O)c2ccc(C)c(C)c2N)c1] | Blue solid | 120-122° C. |
| 10 | 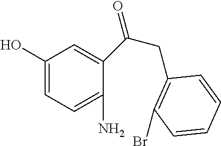 [SMILES: Nc1ccc(O)cc1C(=O)Cc1ccccc1Br] | Yellow solid | 159-160° C. |

We use substituted aniline and substituted o-bromobenzonitrile as raw materials for the synthesis route, which react in the presence of boron trichloride and aluminum trichloride to obtain substituted 1-(2-aminophenyl)-2-(2-bromophenyl)ethane-1-one as a synthesis intermediate of the dibenzodiazepine compound, greatly simplifying the synthesis of such compounds.

### Example 1

### Synthesis of 6,7-dimethyl-5,11-dihydro-10H-dibenzo[b,f]aza-10-one (Compound 11)

Under nitrogen protection, a syringe was used to measure 20 mL of xylene solution of boron trichloride and add to a 100 mL three-necked bottle. Then the three-necked bottle was placed in an ice water bath to cool down and stir, and 10 mL of anhydrous dichloromethane was added. A mixed solution of 2.42 g (20 mmol) of 2,3-dimethylaniline in anhydrous 1,2-dichloroethane (10 mL) was prepared. When the temperature of the solution in the three-necked bottle dropped to 0-5° C., the mixed solution was slowly added dropwise to the xylene solution of boron trichloride using a constant pressure dropping funnel. Then, 5.88 g (30 mmol) of o-bromocyanobenzyl chloride and 2.93 g (22 mmol) of anhydrous aluminum trichloride were added in sequence to obtain the reaction solution, which was heated up to 85° C. and stirred for 20 hours. After confirming the complete reaction on the thin layer chromatography plate, the reaction solution was allowed to stand and cooled to 0° C. Then, 2N HCl was slowly added dropwise for acidification until no bubbles were generated. The mixed solution was refluxed at 80° C. for 30 minutes, extracted with dichloromethane, washed with 1M NaOH, dried, performed rotary evaporation, and purified by silica gel column chromatography to obtain compound 1, 2.57 g green solid, mp (PE/EA): 116-117° C., with a yield of 41%. ¹H NMR (400 MHz, CDCl₃) δ 7.68 (d, J=8.3 Hz, 1H), 7.60 (d, J=8.0 Hz, 1H), 7.29 (t, J=7.4 Hz, 1H), 7.24 (d, J=7.6 Hz, 1H), 7.15 (t, J=6.6 Hz, 1H), 6.56 (d, J=8.3 Hz, 1H), 6.47 (s, 1H), 4.45 (s, 2H), 2.32 (s, 3H), 2.07 (s, 3H). ¹³C NMR (101 MHz, CDCl₃) δ 198.35, 149.39, 143.09, 136.12, 132.75, 131.76, 128.50, 127.49, 125.28, 121.30, 118.06, 115.47, 77.40, 77.08, 76.76, 46.42, 21.25, 12.39.

Under nitrogen protection, compound 1 (111 mg, 0.35 mmol), Pd(OAc)₂(4 mg, 0.017 mmol), BINAP (17.4 mg, 0.027 mmol), and ground K₃PO₄ (150 mg, 0.68 mmol) were added to a 50 mL four-necked bottle. Then, 3.5 mL of toluene and 1.5 mL of water were added to obtain a red suspension. The suspension was heated up to 130° C. and stirred for 5 hours. After confirming the complete reaction on the thin layer chromatography plate, the reaction solution was allowed to stand and cool, and extracted with water and dichloromethane. The organic phase was dried over anhydrous sodium sulfate, performed rotary evaporation, and purified by silica gel column chromatography to obtain a yellow solid. The dry weight was 66.4 mg, mp (PE/EA): 147-149° C., and the total yield was 33%. ¹H NMR (400 MHz, CDCl₃) δ 7.84 (d, J=8.2 Hz, 1H), 7.32 (d, J=7.5 Hz, 1H), 7.20 (d, J=7.5 Hz, 1H), 7.16 (dd, J=15.6, 8.2 Hz, 1H), 7.03 (d, J=7.7 Hz, 1H), 6.79 (d, J=8.2 Hz, 1H), 6.76 (d, J=14.1 Hz, 1H), 3.82 (s, 2H), 2.40 (s, 3H), 2.39 (s, 3H). ¹³C NMR (101 MHz, CDCl₃) δ 189.67, 144.94, 142.16, 141.49, 129.66, 127.89, 127.49, 125.04, 122.77, 122.03, 121.53, 120.04, 77.38, 77.06, 76.74, 49.14, 21.69, 13.55.

### Example 2

### Synthesis of 6,7-dimethyl-5,11-dihydro-10H-dibenzo[b,f]aza-10-ol (Compound 11a)

Compound 11 (0.35 g, 1.48 mmol) was added to a 100 mL flat-bottomed single-necked bottle, then 3 mL of water and 12 mL of methanol were added to obtain a yellow suspension, which was stirred at room temperature. Then, 0.152 g (4.02 mmol) of sodium borohydride was added in batches to the above-mentioned suspension to obtain a yellow transparent reaction solution, which was heated up to 45° C. and stirred for 1 hour. After confirming the complete reaction on the thin layer chromatography plate, 30 mL of acetone was added to the reaction solution to quench it, resulting in a colorless transparent solution. The solution was then performed rotary evaporation and 30 mL of water was added to the residual solution. The residual solution was filtered, and the filter cake was dried under vacuum at 50° C. to obtain 0.31 g gray white solid, mp (H₂O): 126-127° C. The yield was 89.0%. ¹H NMR (400 MHz, CDCl₃) δ 7.20-7.07 (m, 3H), 6.94-6.83 (m, 2H), 6.74 (d, J=7.7 Hz, 1H), 6.07 (s, 1H), 5.14 (t, J=5.4 Hz, 1H), 3.34-3.22 (m, 2H), 2.31 (d, J=11.8 Hz, 6H).

### Example 3

### Synthesis of 7,8-dimethyl-5,11-dihydro-10H-dibenzo[b,f]aza-10-one (Compound 12)

Under nitrogen protection, a syringe was used to measure 25.3 mL of xylene solution of boron trichloride and add to a 100 mL three-necked bottle. Then the three-necked bottle was placed in an ice water bath to cool down and stir. A mixed solution of 2.79 g (23 mmol) of 3,4-dimethylaniline in 1,2-dichloroethane (25 mL) was prepared. When the temperature of the solution in the three-necked bottle dropped to 0-5° C., the mixed solution was slowly dripped to the xylene solution of boron trichloride using a constant pressure dropping funnel. Then, 9.02 g (46 mmol) of o-bromocyanobenzyl chloride and 3.37 g (25.3 mmol) of anhydrous aluminum trichloride were added in sequence to obtain the reaction solution, which was heated up to 85° C. and stirred for 20 hours. After confirming the complete reaction on the thin layer chromatography plate, the reaction solution was allowed to stand and cooled to 0° C. Then, 2N HCl was slowly added dropwise for acidification until no bubbles were generated. The mixed solution was refluxed at 80° C. for 30 minutes, extracted with dichloromethane, washed with 1M NaOH, dried, performed rotary evaporation, and purified by silica gel column chromatography to obtain compound 2, 1.97 g orange solid, mp (PE/EA): 108-109° C., with a yield of 27%. ¹H NMR (400 MHz, CDCl₃) δ 7.61 (d, J=4.0 Hz, 1H), 7.59 (s, 1H), 7.29 (t, J=7.3 Hz, 1H), 7.24 (d, J=6.3 Hz, 1H), 7.15 (t, J=8.1 Hz, 1H), 6.49 (s, 1H), 4.42 (s, 2H), 2.21 (s, 3H), 2.20 (s, 3H). ¹³C NMR (101 MHz, CDCl₃) δ 197.81, 149.12, 144.83, 135.98, 132.75, 131.75, 131.23, 128.55, 127.49, 125.26, 124.30, 118.26, 115.81, 77.38, 77.06, 76.74, 46.30, 20.28, 18.98.

Under nitrogen protection, compound 2 (111 mg, 0.35 mmol), Pd(OAc)₂(4 mg, 0.017 mmol), BINAP (17.4 mg, 0.027 mmol), and ground K₃PO₄(150 mg, 0.68 mmol) were added to a 50 mL four-necked bottle. Then, 3.5 mL of toluene and 1.5 mL of water were added to obtain a red suspension. The suspension was heated up to 130° C. and stirred for 5 hours. After confirming the complete reaction on the thin layer chromatography plate, the reaction solution was allowed to stand and cool, and extracted with water and dichloromethane. The organic phase was dried over anhydrous sodium sulfate, performed rotary evaporation, and purified by silica gel column chromatography to obtain compound 12, a green solid. The dry weight was 61.47 mg, mp (PE/EA): 219-220° C., and the total yield was 20%. ¹H NMR (400 MHz, CDCl₃) δ 7.78 (s, 1H), 7.30 (d, J=7.4 Hz, 1H), 7.20 (t, J=7.4 Hz, 1H), 7.12 (t, J=7.3 Hz, 1H), 7.00 (d, J=7.8 Hz, 1H), 6.81 (s, 1H), 6.45 (s, 1H), 3.80 (s, 2H), 2.28 (s, 3H), 2.21 (s, 3H). ¹³C NMR (101 MHz, CDCl₃) δ 189.73, 144.89, 143.66, 141.89, 130.55, 129.93, 128.21, 127.52, 124.54, 124.03, 122.25, 120.04, 118.66, 77.38, 77.06, 76.74, 49.43, 20.07, 18.57.

### Example 4

### Synthesis of 3-fluoro-6,7-dimethyl-5,11-dihydro-10H-dibenzo[b,f]aza-10-one (Compound 13)

Under nitrogen protection, a syringe was used to measure 25.3 mL of xylene solution of boron trichloride and add to a 100 mL three-necked bottle. Then the three-necked bottle was placed in an ice water bath to cool down and stir. A mixed solution of 2.79 g (23 mmol) of 2,3-dimethylaniline in 1,2-dichloroethane (25 mL) was prepared. When the temperature of the solution in the three-necked bottle dropped to 0-5° C., the mixed solution was slowly dripped to the xylene solution of boron trichloride using a constant pressure dropping funnel. Then, 9.85 g (46 mmol) of 2-bromo-4-fluorophenylacetonitrile and 3.37 g (25.3 mmol) of anhydrous aluminum trichloride were added in sequence to obtain the reaction solution, which was heated up to 85° C. and stirred for 20 hours. After confirming the complete reaction on the thin layer chromatography plate, the reaction solution was allowed to stand and cooled to 0° C. Then, 2N HCl was slowly added dropwise for acidification until no bubbles were generated. The mixed solution was refluxed at 80° C. for 30 minutes, extracted with dichloromethane, washed with 1M NaOH, dried, performed rotary evaporation, and purified by silica gel column chromatography to obtain compound 3, 1.54 g white solid, mp (PE/EA): 112-114° C., with a yield of 20%. ¹H NMR (400 MHz, CDCl₃) δ 7.67 (d, J=8.3 Hz, 1H), 7.35 (dd, J=8.3, 2.6 Hz, 1H), 7.21 (dd, J=8.4, 6.0 Hz, 1H), 7.02 (td, J=8.3, 2.5 Hz, 1H), 6.56 (d, J=8.3 Hz, 1H), 6.48 (s, 1H), 4.41 (s, 2H), 2.32 (s, 3H), 2.07 (s, 3H). ¹³C NMR (101 MHz, CDCl₃) δ 198.07, 162.72, 160.24, 149.49, 143.31, 132.53, 132.07, 128.39, 125.22, 121.43, 120.14, 119.89, 118.18, 115.38, 114.80, 114.59, 77.48, 77.16, 76.84, 45.57, 21.32, 12.46.

Under nitrogen protection, compound 3 (117 mg, 0.35 mmol), Pd(OAc)₂ (4 mg, 0.017 mmol), BINAP (17.4 mg, 0.027 mmol), and ground K₃PO₄ (150 mg, 0.68 mmol) were added to a 50 mL four-necked bottle. Then, 3.5 mL of toluene and 1.5 mL of water were added to obtain a red suspension. The suspension was heated up to 130° C. and stirred for 5 hours. After confirming the complete reaction on the thin layer chromatography plate, the reaction solution was allowed to stand and cool, and extracted with water and dichloromethane. The organic phase was dried over anhydrous sodium sulfate, performed rotary evaporation, and purified by silica gel column chromatography to obtain compound 13, a green solid. The dry weight was 66.9 mg, mp (PE/EA): 154-155° C., and the total yield was 15%. ¹H NMR (400 MHz, CDCl₃) δ 7.83 (d, J=8.2 Hz, 1H), 7.27 (s, 1H), 7.26-7.22 (m, 1H), 6.85 (ddd, J=13.9, 9.9, 5.3 Hz, 2H), 6.79-6.64 (m, 2H), 3.79 (s, 2H), 2.39 (s, 6H). ¹³C NMR (101 MHz, CDCl₃) δ 189.47, 144.39, 142.75, 142.41, 130.91, 127.94, 122.86, 122.05, 120.92, 112.02, 111.81, 107.14, 106.90, 77.38, 77.06, 76.74, 48.37, 21.70, 13.54.

### Example 5

### Synthesis of 5,11-dihydro-10H-dibenzo[b,f]aza-10-one (Compound 14)

Under nitrogen protection, a syringe was used to measure 26.5 mL of xylene solution of boron trichloride and add to a 100 mL three-necked bottle. Then the three-necked bottle was placed in an ice water bath to cool down and stir. A mixed solution of 2.24 g (24 mmol) of aniline in 1,2-dichloroethane (25 mL) was prepared. When the temperature of the solution in the three-necked bottle dropped to 0-5° C., the mixed solution was slowly dripped to the xylene solution of boron trichloride using a constant pressure dropping funnel. Then, 9.43 g (48 mmol) of o-bromocyanobenzyl chloride and 3.53 g (26.5 mmol) of anhydrous aluminum trichloride were added in sequence to obtain the reaction solution, which was heated up to 85° C. and stirred for 20 hours. After confirming the complete reaction on the thin layer chromatography plate, the reaction solution was allowed to stand and cooled to 0° C. Then, 2N HCl was slowly added dropwise for acidification until no bubbles were generated. The mixed solution was refluxed at 80° C. for 30 minutes, extracted with dichloromethane, washed with 1M NaOH, dried, performed rotary evaporation, and purified by silica gel column chromatography to obtain compound 4, 1.88 g yellow solid, mp (PE/EA): 88-90° C., with a yield of 27%. ¹H NMR (400 MHz, CDCl₃) δ 7.83 (d, J=8.1 Hz, 1H), 7.57 (d, J=8.0 Hz, 1H), 7.28 (s, 1H), 7.24 (s, 1H), 7.20 (d, J=7.4 Hz, 1H), 7.12 (t, J=7.6 Hz, 1H), 6.66 (d, J=9.6 Hz, 1H), 6.63 (s, 1H), 4.41 (s, 2H).

Under nitrogen protection, compound 4 (100.6 mg, 0.35 mmol), Pd(OAc)₂ (4 mg, 0.017 mmol), BINAP (17.4 mg, 0.027 mmol), and ground K₃PO₄(150 mg, 0.68 mmol) were added to a 50 mL four-necked bottle. Then, 3.5 mL of toluene and 1.5 mL of water were added to obtain a red suspension. The suspension was heated up to 130° C. and stirred for 5 hours. After confirming the complete reaction on the thin layer chromatography plate, the reaction solution was allowed to stand and cool, and extracted with water and dichloromethane. The organic phase was dried over anhydrous sodium sulfate, performed rotary evaporation, and purified by silica gel column chromatography to obtain a green solid. The dry weight was 58.5 mg, mp (PE/EA): 138-140° C., and the total yield was 22%. ¹H NMR (400 MHz, CDCl₃) δ 8.03 (dd, J=8.0, 1.3 Hz, 1H), 7.46-7.37 (m, 1H), 7.31 (d, J=7.4 Hz, 1H), 7.22 (td, J=7.6, 1.4 Hz, 1H), 7.18-7.12 (m, 1H), 7.05 (s, 1H), 7.03 (d, J=7.7 Hz, 1H), 6.94 (t, J=7.5 Hz, 1H), 6.68 (s, 1H), 3.84 (s, 2H).

### Example 6

### Synthesis of 6-chloro-5,11-dihydro-10H-dibenzo[b,f]aza-10-one (Compound 15)

Under nitrogen protection, a syringe was used to measure 25.3 mL of xylene solution of boron trichloride and add to a 100 mL three-necked bottle. Then the three-necked bottle was placed in an ice water bath to cool down and stir. A mixed solution of 2.92 g (23 mmol) of o-chloroaniline in 1,2-dichloroethane (25 mL) was prepared. When the temperature of the solution in the three-necked bottle dropped to 0-5° C., the mixed solution was slowly dripped to the xylene solution of boron trichloride using a constant pressure dropping funnel. Then, 9.02 g (46 mmol) of o-bromocyanobenzyl chloride and 3.37 g (25.3 mmol) of anhydrous aluminum trichloride were added in sequence to obtain the reaction solution, which was heated up to 85° C. and stirred for 20 hours. After confirming the complete reaction on the thin layer chromatography plate, the reaction solution was allowed to stand and cooled to 0° C. Then, 2N HCl was slowly added dropwise for acidification until no bubbles were generated. The mixed solution was refluxed at 80° C. for 30 minutes, extracted with dichloromethane, washed with 1M NaOH, dried, performed rotary evaporation, and purified by silica gel column chromatography to obtain compound 5, 2.0 g cyan solid, mp (PE/EA): 133-135° C., with a yield of 27%. ¹H NMR (400 MHz, CDCl₃) δ 7.83 (dd, J=8.2, 1.2 Hz, 1H), 7.61 (dd, J=8.0, 0.9 Hz, 1H), 7.45 (dd, J=7.7, 1.3 Hz, 1H), 7.31 (td, J=7.5, 1.0 Hz, 1H), 7.23 (dd, J=7.6, 1.6 Hz, 1H), 7.17 (td, J=7.8, 1.7 Hz, 1H), 6.65 (t, J=7.9 Hz, 1H), 4.46 (s, 2H). ¹³C NMR (101 MHz, CDCl₃) δ 198.25, 146.86, 135.47, 134.29, 132.93, 131.83, 129.94, 128.91, 127.68, 125.32, 120.93, 118.46, 115.45, 46.65.

Under nitrogen protection, compound 5 (114 mg, 0.35 mmol), Pd(OAc)₂ (4 mg, 0.017 mmol), BINAP (17.4 mg, 0.027 mmol), and ground K₃PO₄ (150 mg, 0.68 mmol) were added to a 50 mL four-necked bottle. Then, 3.5 mL of toluene and 1.5 mL of water were added to obtain a red suspension. The suspension was heated up to 130° C. and stirred for 5 hours. After confirming the complete reaction on the thin layer chromatography plate, the reaction solution was allowed to stand and cool, and extracted with water and dichloromethane. The organic phase was dried over anhydrous sodium sulfate, performed rotary evaporation, and purified by silica gel column chromatography to obtain a chartreuse solid. The dry weight was 66.2 mg, mp (PE/EA): 115-116° C., and the total yield was 21%. ¹H NMR (400 MHz, CDCl₃) δ 7.95 (d, J=8.0 Hz, 1H), 7.57 (d, J=7.7 Hz, 1H), 7.52 (s, 1H), 7.33 (d, J=7.4 Hz, 1H), 7.21 (dd, J=15.5, 8.2 Hz, 1H), 7.12 (d, J=7.7 Hz, 1H), 6.87 (t, J=7.9 Hz, 1H), 3.84 (s, 2H). ¹³C NMR (101 MHz, CDCl₃) δ 188.67, 142.08, 140.54, 133.38, 129.85, 129.65, 127.85, 125.81, 125.59, 124.46, 122.24, 120.14, 118.98, 77.38, 77.06, 76.74, 49.04.

### Example 7

### Synthesis of 7-chloro-5,11-dihydro-10H-dibenzo[b,f]aza-10-one (Compound 16)

Under nitrogen protection, a syringe was used to measure 25.3 mL of xylene solution of boron trichloride and add to a 100 mL three-necked bottle. Then the three-necked bottle was placed in an ice water bath to cool down and stir. A mixed solution of 2.92 g (23 mmol) of m-chloroaniline in 1,2-dichloroethane (25 mL) was prepared. When the temperature of the solution in the three-necked bottle dropped to 0-5° C., the mixed solution was slowly dripped to the xylene solution of boron trichloride using a constant pressure dropping funnel. Then, 9.02 g (46 mmol) of o-bromocyanobenzyl chloride and 3.37 g (25.3 mmol) of anhydrous aluminum trichloride were added in sequence to obtain the reaction solution, which was heated up to 85° C. and stirred for 20 hours. After confirming the complete reaction on the thin layer chromatography plate, the reaction solution was allowed to stand and cooled to 0° C. Then, 2N HCl was slowly added dropwise for acidification until no bubbles were generated. The mixed solution was refluxed at 80° C. for 30 minutes, extracted with dichloromethane, washed with 1M NaOH, dried, performed rotary evaporation, and purified by silica gel column chromatography to obtain compound 6, 2.0 g green solid, mp (PE/EA): 115-117° C., with a yield of 27%. ¹H NMR (400 MHz, CDCl₃) δ 7.78 (d, J=8.6 Hz, 1H), 7.61 (dd, J=8.0, 1.1 Hz, 1H), 7.30 (td, J=7.5, 1.2 Hz, 1H), 7.23 (dd, J=7.6, 1.7 Hz, 1H), 7.16 (td, J=7.8, 1.8 Hz, 1H), 6.67 (d, J=1.9 Hz, 1H), 6.64 (dd, J=8.6, 2.0 Hz, 1H), 6.33 (s, 1H), 4.40 (s, 2H). ¹³C NMR (101 MHz, CDCl₃) δ 198.25, 146.86, 135.47, 134.29, 132.93, 131.83, 129.94, 128.91, 127.68, 125.32, 120.93, 118.46, 115.45, 46.65.

Under nitrogen protection, compound 6 (114 mg, 0.35 mmol), Pd(OAc)₂ (4 mg, 0.017 mmol), BINAP (17.4 mg, 0.027 mmol), and ground K₃PO₄(150 mg, 0.68 mmol) were added to a 50 mL four-necked bottle. Then, 3.5 mL of toluene and 1.5 mL of water were added to obtain a red suspension. The suspension was heated up to 130° C. and stirred for 5 hours. After confirming the complete reaction on the thin layer chromatography plate, the reaction solution was allowed to stand and cool, and extracted with water and dichloromethane. The organic phase was dried over anhydrous sodium sulfate, performed rotary evaporation, and purified by silica gel column chromatography to obtain compound 16, a green solid. The dry weight was 62.9 mg, mp (PE/EA): 243-245° C., and the total yield was 20%. ¹H NMR (400 MHz, CDCl₃) δ 7.96 (d, J=8.6 Hz, 1H), 7.31 (d, J=7.4 Hz, 1H), 7.23 (d, J=7.5 Hz, 1H), 7.18 (t, J=7.2 Hz, 1H), 7.06 (d, J=1.4 Hz, 1H), 7.04 (d, J=8.0 Hz, 1H), 6.90 (dd, J=8.6, 1.5 Hz, 1H), 6.61 (s, 1H), 3.82 (s, 2H). ¹³C NMR (101 MHz, CDCl₃) δ 147.11, 140.83, 132.25, 130.06, 127.85, 125.40, 124.04, 120.00, 119.13, 118.68, 77.38, 77.06, 76.74, 49.18.

### Example 8

### Synthesis of 8-methoxy-5,11-dihydro-10H-dibenzo[b,f]aza-10-one and 8-hydroxy-5,11-dihydro-10H-dibenzo[b,f]aza-10-one (compounds 17 and 20)

Under nitrogen protection, a syringe was used to measure 25.3 mL of xylene solution of boron trichloride and add to a 100 mL three-necked bottle. Then the three-necked bottle was placed in an ice water bath to cool down and stir. A mixed solution of 2.83 g (23 mmol) of p-methoxyaniline in 1,2-dichloroethane (25 mL) was prepared. When the temperature of the solution in the three-necked bottle dropped to 0-5° C., the mixed solution was slowly dripped to the xylene solution of boron trichloride using a constant pressure dropping funnel. Then, 9.02 g (46 mmol) of o-bromocyanobenzyl chloride and 3.37 g (25.3 mmol) of anhydrous aluminum trichloride were added in sequence to obtain the reaction solution, which was heated up to 85° C. and stirred for 20 hours. After confirming the complete reaction on the thin layer chromatography plate, the reaction solution was allowed to stand and cooled to 0° C. Then, 2N HCl was slowly added dropwise for acidification until no bubbles were generated. The mixed solution was refluxed at 80° C. for 30 minutes, extracted with dichloromethane, washed with 1M NaOH, dried, performed rotary evaporation, and purified by silica gel column chromatography to obtain compound 7, 1.49 g green solid, mp (PE/EA): 79-80° C., with a yield of 20%. ¹H NMR (400 MHz, CDCl₃) δ 7.61 (d, J=8.0 Hz, 1H), 7.31 (d, J=2.8 Hz, 1H), 7.30-7.27 (m, 1H), 7.24 (d, J=1.9 Hz, 1H), 7.16 (td, J=7.9, 2.0 Hz, 1H), 7.00 (dd, J=9.0, 2.8 Hz, 1H), 6.72 (d, J=9.0 Hz, 1H), 4.43 (s, 2H), 3.79 (s, 3H). ¹³C NMR (101 MHz, CDCl₃) δ 198.27, 150.42, 145.18, 135.70, 132.93, 131.70, 128.82, 127.71, 125.30, 123.69, 119.19, 117.62, 113.71, 56.23, 46.68.

In addition, a product with higher polarity was obtained as compound 10, 1.19 g yellow solid, mp (PE/EA): 159-160° C., with a yield of 17%. ¹H NMR (400 MHz, CDCl₃) δ 7.60 (d, J=7.8 Hz, 1H), 7.31 (dd, J=15.5, 5.1 Hz, 2H), 7.23 (d, J=7.4 Hz, 1H), 7.16 (t, J=7.5 Hz, 1H), 6.92 (dd, J=8.9, 2.6 Hz, 1H), 6.61 (d, J=8.8 Hz, 1H), 5.93 (s, 1H), 4.52-4.35 (m, 2H). ¹³C NMR (101 MHz, CDCl₃) δ 197.95, 145.45, 135.53, 132.81, 131.75, 128.74, 127.57, 125.24, 124.10, 118.87, 17.52, 115.78, 77.38, 77.06, 76.74, 46.48.

Under nitrogen protection, the above-mentioned compound 7 (112 mg, 0.35 mmol), Pd(OAc)₂ (4 mg, 0.017 mmol), BINAP (17.4 mg, 0.027 mmol), and ground K₃PO₄ (150 mg, 0.68 mmol) were added to a 50 mL four-necked bottle. Then, 3.5 mL of toluene and 1.5 mL of water were added to obtain a red suspension. The suspension was heated up to 130° C. and stirred for 5 hours. After confirming the complete reaction on the thin layer chromatography plate, the reaction solution was allowed to stand and cool, and extracted with water and dichloromethane. The organic phase was dried over anhydrous sodium sulfate, performed rotary evaporation, and purified by silica gel column chromatography to obtain compound 17, a green solid. The dry weight was 54.7 mg, mp (PE/EA): 176-177° C., and the total yield was 13%. ¹H NMR (400 MHz, CDCl₃) δ 7.50 (d, J=2.9 Hz, 1H), 7.31 (d, J=7.4 Hz, 1H), 7.20 (t, J=7.0 Hz, 1H), 7.13 (t, J=7.2 Hz, 1H), 7.06 (dd, J=8.9, 3.0 Hz, 1H), 7.03 (d, J=7.8 Hz, 1H), 6.97 (d, J=8.9 Hz, 1H), 6.47 (s, 1H), 3.83 (s, 2H), 3.80 (s, 3H). ¹³C NMR (101 MHz, CDCl₃) δ 189.45, 152.93, 142.09, 141.41, 129.90, 127.61, 124.67, 124.43, 123.75, 123.33, 120.97, 118.70, 110.82, 77.38, 77.06, 76.74, 55.79, 49.23.

Under nitrogen protection, the above-mentioned compound 10 (112 mg, 0.35 mmol), Pd(OAc)₂ (4 mg, 0.017 mmol), BINAP (17.4 mg, 0.027 mmol), and ground K₃PO₄ (150 mg, 0.68 mmol) were added to a 50 mL four-necked bottle. Then, 3.5 mL of toluene and 1.5 mL of water were added to obtain a red suspension. The suspension was heated up to 130° C. and stirred for 5 hours. After confirming the complete reaction on the thin layer chromatography plate, the reaction solution was allowed to stand and cool, and extracted with water and dichloromethane. The organic phase was dried over anhydrous sodium sulfate, performed rotary evaporation, and purified by silica gel column chromatography to obtain compound 20, a yellow solid. The dry weight was 69.5 mg, mp (PE/EA): 157-158° C., and the total yield was 15%. ¹H NMR (400 MHz, CDCl₃) δ 7.57 (d, J=2.9 Hz, 1H), 7.30 (d, J=7.3 Hz, 1H), 7.21 (t, J=7.5 Hz, 1H), 7.13 (t, J=7.0 Hz, 1H), 7.04 (dd, J=13.3, 4.9 Hz, 2H), 6.94 (d, J=8.7 Hz, 1H), 6.45 (s, 1H), 5.82 (s, 1H), 3.82 (s, 2H). MS(ESI)m/z: calcd for C₁₄H₁₁NO₂(M⁺):225.1, Found:226.1.

### Example 9

### Synthesis of 3-methoxy-6,7-dimethyl-5,11-dihydro-10H-dibenzo[b,f]aza-10-one (Compound 18)

3-bromo-4-methylbenzyl ether (5 g, 24 mmol, lequiv), NBS (5.13 g, 28.8 mmol, 1.2 equiv), and AIBN (0.08 g, 0.48 mmol, 0.02 equiv) were added to benzene to obtain a suspension. The reaction solution was heated to reflux for 1 hour. After confirming the complete reaction on the thin layer chromatography plate, the reaction solution was allowed to stand and cool, filtered. The filtrate was extracted with water and ethyl acetate, and the organic phase was washed with saturated sodium chloride and water, dried with anhydrous sodium sulfate, and performed rotary evaporation to obtain white solid 3-bromo-4-bromomethylphenyl ether (5.5 g, 19.8 mmol).

3-bromo-4-bromomethylphenyl ether (2 g, 7.20 mmol) was mixed with water (10 mL) and ethanol (20 mL) to obtain a mixed solution, then NaCN (0.4 g, 8.16 mmol) was added. The resulting reaction solution was stirred at 80° C. for 2 hours. After confirming the complete reaction on the thin layer chromatography plate, the reaction solution was allowed to stand and cool, quenched by sodium thiosulfate, and extracted and separated with water and EA. The organic phase was washed with saturated sodium chloride, dried with anhydrous sodium sulfate, and performed rotary evaporation to obtain 1.38 g of white solid 2-bromo-4-methoxyphenylacetonitrile, mp (PE/EA): 62-63° C., and the total yield was 70%. ¹H NMR (400 MHz, CDCl3) δ 7.38 (d, J=8.6 Hz, 1H), 7.14 (d, J=2.6 Hz, 1H), 6.88 (dd, J=8.6, 2.6 Hz, 1H), 3.80 (s, 3H), 3.76 (s, 2H).

Under nitrogen protection, a syringe was used to measure 25.3 mL of xylene solution of boron trichloride and add to a 100 mL three-necked bottle. Then the three-necked bottle was placed in an ice water bath to cool down and stir. A mixed solution of 2.79 g (23 mmol) of 2,3-dimethylaniline in 1,2-dichloroethane (25 mL) was prepared. When the temperature of the solution in the three-necked bottle dropped to 0-5° C., the mixed solution was slowly dripped to the xylene solution of boron trichloride using a constant pressure dropping funnel. Then, 10.3 g (46 mmol) of 2-bromo-4-methoxyphenylacetonitrile and 3.37 g (25.3 mmol) of anhydrous aluminum trichloride were added in sequence to obtain the reaction solution, which was heated up to 85° C. and stirred for 20 hours. After confirming the complete reaction on the thin layer chromatography plate, the reaction solution was allowed to stand and cooled to 0° C. Then, 2N HCl was slowly added dropwise for acidification until no bubbles were generated. The mixed solution was refluxed at 80° C. for 30 minutes, extracted with dichloromethane, washed with 1M NaOH, dried, performed rotary evaporation, and purified by silica gel column chromatography to obtain compound 8, 1.91 g yellow solid, mp (PE/EA): 165-167° C., with a yield of 24%. ¹H NMR (400 MHz, CDCl₃) δ 7.68 (d, J=8.3 Hz, 1H), 7.18-7.13 (m, 1H), 7.12 (s, 1H), 6.84 (dd, J=8.5, 2.5 Hz, 1H), 6.55 (d, J=8.3 Hz, 1H), 6.47 (s, 2H), 4.38 (s, 2H), 3.79 (s, 3H), 2.31 (s, 3H), 2.07 (s, 3H). ¹³C NMR (101 MHz, CDCl₃) δ 198.76, 159.04, 149.34, 143.02, 131.99, 128.42, 127.93, 125.35, 121.28, 118.01, 115.46, 113.68, 77.38, 77.06, 76.74, 55.56, 45.47, 21.24, 12.39.

Under nitrogen protection, compound 8 (121 mg, 0.35 mmol), Pd(OAc)₂(4 mg, 0.017 mmol), BINAP (17.4 mg, 0.027 mmol), and ground K₃PO₄ (150 mg, 0.68 mmol) were added to a 50 mL four-necked bottle. Then, 3.5 mL of toluene and 1.5 mL of water were added to obtain a red suspension. The suspension was heated up to 130° C. and stirred for 5 hours. After confirming the complete reaction on the thin layer chromatography plate, the reaction solution was allowed to stand and cool, and extracted with water and dichloromethane. The organic phase was dried over anhydrous sodium sulfate, performed rotary evaporation, and purified by silica gel column chromatography to obtain compound 18, a yellow solid. The dry weight was 77.9 mg, mp (PE/EA): 131-132° C., and the total yield was 20%. ¹H NMR (400 MHz, CDCl₃) δ 7.82 (d, J=8.2 Hz, 1H), 7.20 (d, J=8.4 Hz, 1H), 6.79 (d, J=8.2 Hz, 1H), 6.72 (dd, =8.4, 2.4 Hz, 1H), 6.68 (s, 1H), 6.55 (d, J=2.3 Hz, 1H), 3.79 (s, 3H), 3.74 (s, 2H), 2.39 (d, J=2.5 Hz, 6H). ¹³C NMR (101 MHz, CDCl₃) δ 189.90, 159.13, 144.73, 142.45, 142.04, 130.48, 127.94, 122.71, 121.93, 121.59, 117.49, 110.81, 105.51, 77.38, 77.06, 76.74, 55.55, 48.20, 21.67, 13.57.

### Example 10

### Synthesis of 2-methoxy-6,7-dimethyl-5,11-dihydro-10H-dibenzo[b,f]aza-10-one (Compound 19)

2-bromo-5-methoxytoluene (5 g, 24 mmol, 1 equiv), NBS (5.13 g, 28.8 mmol, 1.2 equiv), and AIBN (0.08 g, 0.48 mmol, 0.02 equiv) were added to benzene to obtain a suspension. The reaction solution was heated to reflux for 1 hour. After confirming the complete reaction on the thin layer chromatography plate, the reaction solution was allowed to stand and cool, filtered. The filtrate was extracted with water and ethyl acetate, and the organic phase was washed with saturated sodium chloride and water, dried with anhydrous sodium sulfate, and performed rotary evaporation to obtain white solid 2-bromo-5-methoxybenzyl bromide (4.2 g, 15.1 mmol).

2-bromo-5-methoxybenzyl bromide (2 g, 7.20 mmol) was mixed with water (10 mL) and ethanol (20 mL) to obtain a mixed solution. Then NaCN (0.4 g, 8.16 mmol) was added and the resulting reaction solution was stirred at 80° C. for 2 hours. After confirming the complete reaction on the thin layer chromatography plate, the reaction solution was allowed to stand and cool, quenched by sodium thiosulfate, and extracted and separated with water and EA. The organic phase was washed with saturated sodium chloride, dried with anhydrous sodium sulfate, and performed rotary evaporation to obtain 1.3 g of white solid 2-bromo-5-methoxyphenylacetonitrile, mp (PE/EA): 55-57° C., and the total yield was 50%. ¹H NMR (400 MHz, CDCl₃) δ 7.46 (d, J=8.8 Hz, 1H), 7.06 (d, J=2.9 Hz, 1H), 6.77 (dd, J=8.8, 2.9 Hz, 1H), 3.82 (s, 3H), 3.80 (s, 2H).

Under nitrogen protection, a syringe was used to measure 25.3 mL of xylene solution of boron trichloride and add to a 100 mL three-necked bottle. Then the three-necked bottle was placed in an ice water bath to cool down and stir. A mixed solution of 2.79 g (23 mmol) of 2,3-dimethylaniline in 1,2-dichloroethane (25 mL) was prepared. When the temperature of the solution in the three-necked bottle dropped to 0-5° C., the mixed solution was slowly dripped to the xylene solution of boron trichloride using a constant pressure dropping funnel. Then, 10.40 g (46 mmol) of 2-bromo-5-methoxyphenylacetonitrile and 3.37 g (25.3 mmol) of anhydrous aluminum trichloride were added in sequence to obtain the reaction solution, which was heated up to 85° C. and stirred for 20 hours. After confirming the complete reaction on the thin layer chromatography plate, the reaction solution was allowed to stand and cooled to 0° C. Then, 2N HCl was slowly added dropwise for acidification until no bubbles were generated. The mixed solution was refluxed at 80° C. for 30 minutes, extracted with dichloromethane, washed with 1M NaOH, dried, performed rotary evaporation, and purified by silica gel column chromatography to obtain compound 9, 1.92 g blue solid, mp (PE/EA): 120-122° C., with a yield of 24%. ¹H NMR (400 MHz, CDCl₃) δ 7.67 (d, J=8.3 Hz, 1H), 7.47 (d, J=8.8 Hz, 1H), 6.80 (d, J=3.0 Hz, 1H), 6.71 (dd, J=8.8, 3.0 Hz, 1H), 6.56 (s, 1H), 6.51 (d, J=27.4 Hz, 2H), 4.40 (s, 2H), 3.77 (s, 3H), 2.31 (s, 3H), 2.07 (s, 3H). ¹³C NMR (101 MHz, CDCl₃) δ 198.24, 158.90, 149.39, 143.09, 136.97, 133.20, 128.46, 121.27, 118.06, 117.38, 115.69, 115.44, 114.24, 77.38, 77.06, 76.74, 55.44, 46.60, 21.23, 12.38.

Under nitrogen protection, compound 9 (121 mg, 0.35 mmol), Pd(OAc)₂(4 mg, 0.017 mmol), BINAP (17.4 mg, 0.027 mmol), and ground K₃PO₄ (150 mg, 0.68 mmol) were added to a 50 mL four-necked bottle. Then, 3.5 mL of toluene and 1.5 mL of water were added to obtain a red suspension. The suspension was heated up to 130° C. and stirred for 5 hours. After confirming the complete reaction on the thin layer chromatography plate, the reaction solution was allowed to stand and cool, and extracted with water and dichloromethane. The organic phase was dried over anhydrous sodium sulfate, performed rotary evaporation, and purified by silica gel column chromatography to obtain compound 19, a cyan solid. The dry weight was 81.8 mg, mp (PE/EA): 155-157° C., and the total yield was 21%. ¹H NMR (400 MHz, CDCl₃) δ 7.82 (d, J=8.2 Hz, 1H), 6.96 (d, J=8.6 Hz, 1H), 6.84 (d, J=2.5 Hz, 1H), 6.76 (dd, J=8.3, 4.6 Hz, 2H), 6.62 (s, 1H), 3.80 (s, 3H), 3.78 (s, 2H), 2.38 (s, 6H). ¹³C NMR (101 MHz, CDCl₃) δ 189.52, 157.47, 145.44, 142.03, 135.07, 127.87, 126.73, 122.31, 121.83, 121.25, 113.93, 113.29, 77.38, 77.06, 76.74, 55.68, 49.30, 21.62, 13.45.

### Example 11

The ability of compounds to secrete immune factors

### 1. Testing Principle:

After entering the cytoplasm, dsDNA from viruses, bacteria, and dead cells can bind and activate cGAS, further inducing the synthesis of the second messenger molecule cGAMP. CGAMP binds to STING on the endoplasmic reticulum (ER) membrane, activating and translocating STING to ERGIC and Golgi apparatus. After translocation, STING recruits TANK binding enzyme 1 (TBK1) and phosphorylates it, followed by recruitment of interferon regulatory factor 3 (IRF3) transcription factor to promote phosphorylation and dimerization of IRF3 and transfers it to the nucleus to induce the expression of type I interferon. In order to accurately evaluate the biological activity of the target compound, L929 mouse epithelial-like fibroblasts were selected for biological experiments to preliminarily evaluate the mouse-derived STING agonist activity of the target compound.

### 2. Testing Method:

### (1) Cell Culture, Recovery, and Pretreatment

Cell recovery: L929 cell cryovials were removed from the liquid nitrogen tank and immediately placed in a 37° C. constant temperature water bath to be thawed rapidly and gently shaken continuously, while ensuring that the vial opening was above the water surface to prevent water contamination of the sample. After 1 minute, the liquid completely dissolved. The outer wall of the cryovials was wiped with alcohol and transferred to a biosafety cabinet. Then, a sterile pipette tip was use to aspirate the cell cryopreservation solution and slowly add dropwise into a 15 mL centrifuge tube containing 2 mL of 10% FBS+1% streptomycin DMEM medium, pipette up and down 5 times to thoroughly mix the cryopreservation solution with the culture medium, while minimizing the concentration of DMSO in the cryopreservation solution as much as possible and reducing cell damage. The vial opening was sealed with a sealing film, centrifuged at room temperature for 5 minutes at 700 rpm, and then the supernatant was discarded. 1 mL of the above-mentioned fresh culture medium was added to resuspend the cells and transfer them to a 4 mL cell culture bottle, which was gently shaken left and right, while avoiding the formation of bubbles as much as possible during the process. Then the culture bottle was placed in a cell culture incubator at 37° C. and 5% CO₂ for cultivation. During this period, when more than half of the cells adhered to the wall, the medium was changed to continue the cultivation.

Cell culture: when the cell density in the cell culture bottle reaches 90% or more, cells were passaged, and the old culture medium was discarded, cells were washed once with 1 mL PBS, and a pipette was used to aspirate PBS and discard. Then 1 mL of trypsin digestion solution was added, the cell bottle was gently shaked left and right, and placed in the incubator for 2-5 minutes. After observing under the microscope that the cells had become round, 2 mL of new culture medium was added to terminate digestion. The culture bottle was gently slapped to accelerate cell detachment. Then the mixed cells were gently pipetted with a pipette, and the suspension was transferred to a 15 mL centrifuge tube, centrifuged at room temperature for 5 minutes at 700 rpm, and then the supernatant was discarded. 2-3 mL of culture medium was added again to resuspend the cells. 10 μL of cell suspension was transferred and placed on a cell counting chamber for counting. 12-well plates were plated at a cell density of 2.5×10⁵/mL.

Pretreatment: the drugs were prepared uniformly into 10 μg/μL. After 12 hours of cell adhesion to the wall, the drugs were added to 12-well plates at final concentrations of 100 μg/mL and 50 μg/mL. After standing for 6 hours, the cell morphology was observed under a microscope, and then the cells were collected into centrifuge tubes.

(2) Extraction and Reverse Transcription of Total RNA from Cells

The total RNA was extracted from L929 cells. According to the instructions of the RNA extraction kit, the centrifugal column method was used for extraction with 300 μL of lysis solution and 300 μL of diluent.

The RNA extraction steps were as follows: 300 μL of RNA diluent was added to a centrifuge tube, mixed well with a pipette to obtain a suspension, stood at room temperature for 3-5 minutes (heating at 70° C. for 3 minutes can improve RNA yield), centrifuged at maximum speed for 5 minutes, then the supernatant was discarded to let the RNA settle at the bottom of the tube (do not use centrifugation to make the RNA too dry, otherwise it is difficult to dissolve). Then anhydrous ethanol with a volume 0.5 times that of the supernatant was added, pipetted 3-4 times with a pipette to mix thoroughly, and the suspension was transferred to a centrifuge column, centrifuged at 12000-14000 rpm for 1 minute, and the filtrate was discarded. 600 μL of RNA wash solution was added, centrifuged at 12000-14000 rpm for 45 seconds, and the filtrate was discarded. Then 50 μL of DNase I incubation solution (5 μL of 10×DNase I buffer, 5 μL of DNase I, 40 μL of nuclease-free water) was added to the center of the adsorption membrane, and stood at room temperature for 15 minutes to fully lyse. 600 μL of RNA wash solution was added, centrifuged at 4° C. and 12000-14000 rpm for 45 seconds, and the filtrate was discarded. Repeat once and vacuum centrifuge for 2 minutes. The centrifuge column was transferred to the elution tube, 50-200 μL of nuclease-free water was added to the center of the membrane to dissolve the RNA precipitate, stood at room temperature for 2 minutes, centrifuged at 12000-14000 rpm for 1 minute, collected, the OD value at 450 nm wavelength was measured using a microplate reader to quantify RNA concentration, and then the RNA sample was stored at −80° C.

The steps of RNA reverse transcription were as follows: cDNA is synthesized in two steps. After the first step reaction is completed, it is immediately placed on ice for at least 2 minutes, and then performed the second step reaction. After the two-step reaction is completed, cDNA was diluted 10 times to a concentration of 5 ng/μL and stored at −20° C. in a refrigerator.

TABLE III

| Experiment | | |
|---|---|---|
| number | Reaction system | PCR reaction conditions |
| The first step | 1 μg RNA | 70° C. 10 minutes |
| reaction | 2 μL Oligo (dT) | |
| | RNase-free water was | |
| | added to 14 μL | |
| The second | 4 μL 5 × M-MLV Buffer | 30° C. 10 min, 42° C. |
| step reaction | 1 μL dNTP (10 mM) | 60 min 70° C. 15 min, |
| | 0.5 μL RNase Inhibitor | 4° C. 60 min |
| | 0.5 μL of M-MLV | |
| | reverse transcriptase | |
| | 14 μL of the first step | |
| | reaction product | |

### (3) Real Time Fluorescence Quantitative PCR

Gene level testing was performed according to GeneStar's 2×RealStar Green Fast Mixture with ROX instructions and primers for mouse IFNb were designed based on the sequences published in the NCBI database. Using GAPDH as an internal reference gene, the relative expression level of mRNA was calculated using the 2^{-ΔΔCt} method.

TABLE IV

| | Upstream sequence | Downstream sequence |
|---|---|---|
| Name | (5′→3′) | (5′→3′) |
| GAPDH | ATCAAGAAGGTGGTGAAGCA | AGACAACCTGGTCCTCAGTGT |
| IFNb | ATGAGTGGTGGTTGCAGGC | TGACCTTTCAAATGCAGTAGATTC |

TABLE V

| QPCR reaction system | Reaction conditions |
|---|---|
| 5 μL 2 × RealStar Green Fast | 95° C. 10 minutes; 95° C. |
| Mixture with ROX | 15 seconds, |
| 0.2 μL of forward primer (10 μM) | 60° C. 1 minute, 40 cycles |
| 0.2 μL of reverse primer (10 μM) | |
| 2 μL cDNA (10 ng) | |
| 2.6 μL RNase-free H₂O | |

To quickly find small molecule compounds with good activity, we set the sample screening concentrations to 100 μg/mL and 50 μg/mL.

### 3. Test Results

The experimental results showed that the tested compounds significantly induced IFNb gene expression at a concentration of 100 μg/mL, indicating that these compounds have good biological activity. Further testing was conducted at a concentration of 50 μg/mL, and these compounds were still able to significantly induce IFNb gene expression.

The test results for activating the STING pathway to secrete the immune factor IFNβ are as follows:

TABLE VI

| | IFNb mRNA expression | |
|---|---|---|
| | 2-^{ΔΔCt}value | |
| Compound | 100 μg/mL | 50 μg/mL |

| No. | | 1 | 2 | 3 |
|---|---|---|---|---|
| Control | 1 | 1 | 1 | 1 |
| 11 | 25.66 | 16.04 | 17.06 | 10.65 |
| 13 | 2.97 | 2.37 | 2.48 | 3.34 |
| 14 | 2.56 | 7.51 | 6.30 | 10.00 |
| 15 | 1.61 | | | |
| 17 | 1.54 | | | |
| 18 | 8.76 | 6.92 | 6.07 | 5.62 |
| 19 | 9.47 | 1.12 | 1.27 | 1.01 |

### Example 12

### Research on Antiviral Activity

### Experimental Method:

PK-15 ordinary cell line was cultured in DMEM complete medium (10% FBS and 1% streptomycin) at 37° C. and 5% CO₂ constant temperature incubator. The cells were washed once with PBS during passage, digested with trypsin until the cells became round, and digestion was terminated with complete culture medium. Cell fluid was collected and centrifuged at 700 rpm for 5 minutes, then the cells were cultured in DMEM complete medium. Cell cryopreservation: cells were collected and cell cryopreservation solution was added, which was 90% FBS+10% DMSO. The number of cells cryopreserved in each tube is 1.0×10⁷. The tubes were placed in a cryopreservation box, gradually cooled down at −80° C., and stored in liquid nitrogen the next day. Cell recovery: the cells to be recovered were removed from the liquid nitrogen tank and directly placed in a 37° C. water bath, quickly melted. DMEM culture medium was added, centrifuged and resuspended in a suitable complete culture medium for cultivation.

### Flow Cytometry Analysis:

PK15 cells which grew well were inoculated at a density of 1.5×10⁵ into a 24-well plate, PRV-GFP (MOI=0.1) or VSV-GFP (MOI=0.05) were inoculated, and each compound was added separately (final concentration of 50 μg/mL), and cells were collected after 24 hours of interaction. The cells were digested with EDTA and adherent cells were digested into a single cell state. The cells were washed with pre-cooled PBS three times, PK-15 cells were resuspended, the cell sample was filtered through a 200 mesh sieve, and untreated PK-15 cells were used as a control. The cells with green fluorescence were analyzed using LSR Fortessa flow cytometry, and the flow cytometry data was analyzed using FlowJo-v10 software.

### Test Results:

(1) we only tested the compounds 11, 13, 14, 18, and 19 with the highest induction of IFNb gene expression. Flow cytometry results showed that compared with the untreated control group, compounds 11, 13, 14, 18, and 19 significantly reduced the proportion of PRV-GFP infected cells, indicating that compounds 11, 13, 14, 18, and 19 could inhibit the infection of DNA virus PRV on cells.

TABLE VII
Antiviral effects of each ingredient on
PRV-GFP (porcine pseudorabies virus)

| Compound | Infection Rate/% | | |
|---|---|---|---|
| No. | 1 | 2 | 3 |
| Mock | 0 | 0 | 0 |
| PRV+ | 68.3 | 67.6 | 66.1 |
| 11 | 13.1 | 14.2 | 13.5 |
| 13 | 7.06 | 8.55 | 7.42 |
| 14 | 17.1 | 19.5 | 19.8 |
| 18 | 18.3 | 17..8 | 18.1 |
| 19 | 51 | 55.7 | 50.2 |

(2) Flow cytometry results showed that compared with the untreated control group, compounds 11, 13, 14, 18, and 19 significantly reduced the proportion of VSV-GF infected cells, indicating that compounds 11, 13, 14, 18, and 19 could inhibit the infection of RNA virus VSV on cells.

TABLE VIII
Validation of antiviral effects of each ingredient
on VSV-GFP (vesicular stomatitis virus)

| Compound | Infection Rate/% | | |
|---|---|---|---|
| No. | 1 | 2 | 3 |
| Mock | 0 | 0 | 0 |
| VSV+ | 99.6 | 99.1 | 98.7 |
| 11 | 40 | 31.1 | 33.7 |
| 13 | 94.3 | 92.7 | 92.1 |
| 14 | 76.1 | 66.5 | 65.8 |
| 18 | 13.9 | 12.5 | 12.9 |
| 19 | 99 | 91.1 | 90.5 |

RNA Extraction and Reverse Transcription (Validated by qPCR Using Influenza Virus)

Following the instructions of Promega's Eastep Super total RNA extraction kit. Specific steps:
1) sample treatment: the cultured cells were washed once with cold PBS and 300 μL of RNA lysis solution was added to lyse the cells. The cell lysis solution was transferred into a 1.5 mL EP tube without RNase or DNase.
2) RNA diluent was added to the sample lysate, mixed well, stood at room temperature for 5 minutes, centrifuged at maximum speed for 5 minutes, and the supernatant was carefully aspirated.
3) Then anhydrous ethanol with a volume 0.5 times that of the supernatant was added, pipetted 3-4 times to mix well.
4) A centrifuge column and a collection tube were installed, the mixture was transferred to the centrifuge column, centrifuged at 12000 rpm for 1 minute, and the filtrate was discarded.
5) 600 μL of RNA wash solution was added, centrifuged at 12000 rpm for 1 minute, and the filtrate was discarded.
6) 50 μL of DNase I incubation solution (10 DNase I buffer 5 μL+DNase I 5 μL+nuclease-free water 40 μL) were added to the center of the adsorption membrane, and stood at room temperature for 15 minutes.
7) 600 μL of RNA wash solution was added, centrifuged at 12000 rpm for 1 minute, and the filtrate was discarded.
8) The centrifuge column was reinstalled onto the collection tube and centrifuged at 12000 rpm for 2 minutes.
9) The centrifuge column was transferred to a new centrifuge tube, 30 μL of DNase or RNase-free water was added dropwise, stood at room temperature for 2 minutes, centrifuged at 12000 rpm for 1 minute, and RNA was stored in a −80° C. refrigerator.

Using reverse transcriptase M-MLV for reverse transcription, the specific steps are as follows:

1) The Following Mixed Solution of Templates and Primers were Prepared in the PCR Tube:

RNA template 1 μg Random and Oligo (dT) Primers 2 μL RNase free H₂O was added to 12 μL. 2) The mixed solution was incubated at 70° C. for 10 minutes and rapidly cooled on ice for 2 minutes, then the following reverse transcription system was added: 5×M-MLV Buffer 4 μL dNTP Mixture (10 mM each) 1 μL RNase Inhibitor (40 U/μL) 0.5 μL RTase M-MLV (RNase H-) 0.5 μL RNase free H₂O was added to 20 μL. Reverse transcription program: 30° C. for 10 minutes, 42° C. for 1 hour, 70° C. for 15 minutes, cDNA was frozen in a −20° C. refrigerator.

### 2) Real Time Fluorescence Quantitative PCR

Real Time PCR reaction solution (10 μL) was prepared according to the following composition: SYBR reagent 5 μL, upstream primer (10 μM) 0.15 μL, downstream primer (10 μM) 0.15 μL, cDNA 2 μL, RNase free H₂O 2.7 μL, and PCR reaction procedure was as follows: pre-denaturation at 95° C. for 10 minutes, denaturation at 95° C. for 10 seconds, annealing at 60° C. for 30 seconds, 40 cycles, extension at 72° C. for 30 seconds, melting curve analysis at 95° C. for 15 seconds, 60° C. for 1 minute, and 95° C. for 1 second.

TABLE IX
Effects of each compound on IAV+ (influenza virus) replication

| | GAPDH (glyceraldehyde-3- | | | M1 (M gene of influenza virus) | | |
|---|---|---|---|---|---|---|
| Compound | phosphate dehydrogenase) Ct value | | | Ct value | | |
| No. | 1 | 2 | 3 | 1 | 2 | 3 |
| Mock | 15.3 | 14.59 | 14.27 | 30.6 | 33.05 | 33.59 |
| IAV+ | 15.44 | 15.05 | 14.57 | 18.9 | 18.62 | 18.48 |
| 11 | 15.14 | 14.86 | 14.81 | 18.71 | 18.62 | 18.68 |
| 13 | 15.24 | 14.87 | 14.71 | 19.45 | 20.25 | 19.66 |
| 14 | 15.21 | 15.11 | 15.37 | 18.99 | 19.52 | 19.69 |
| 18 | 14.72 | 14.30 | 14.75 | 19.52 | 19.47 | 19.51 |
| 19 | 15.21 | 14.70 | 14.69 | 21.37 | 21.39 | 21.49 |

The above-mentioned embodiments are only preferred technical solutions of the present invention and should not be considered as limitations on the present invention. The embodiments and features in the embodiments of the present application can be combined arbitrarily without conflict. The scope of protection of the present invention shall be the technical solution described in the claims, including equivalent alternative solutions to the technical features of the technical solution described in the claims. Equivalent substitution and improvement within this scope are also within the scope of protection of the present invention.

What is claimed is:

1. A dibenzodiazepine compound, characterized in that the dibenzodiazepine compound is a compound of formula I or a stereoisomer thereof, or a pharmaceutically acceptable salt or solvate thereof: 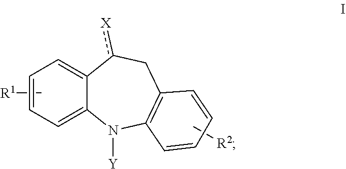
wherein,
X is hydrogen, oxygen, sulfur, C₁₋₆ alkoxy, and C₁₋₆ alkylmercapto;
Y is hydrogen, alkyl, carbamoyl, N—C₁₋₆ alkyl-substituted carbamoyl, acetyl, or other acyls containing 3-10 carbons;
wherein, R¹ and R² are respectively 0-5 different substituents optionally existing on their respective benzene rings, and are independently selected from the following groups: hydrogen, halogen, C₁₋₆ alkyl, hydroxyl, C₁₋₆ haloalkyl, C₁₋₆ alkoxy, amino, amino mono- or di-substituted with C₁₋₆ alkyl, carboxyl, acetate, amide, and phenyl.

2. A dibenzodiazepine compound according to claim 1, wherein the dibenzodiazepine compound is a compound of formula II or a stereoisomer thereof, or a pharmaceutically acceptable salt or solvate thereof: 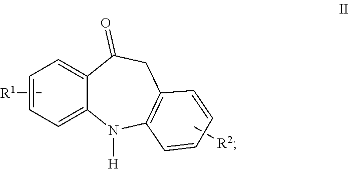
wherein, R¹ and R² are respectively 0-5 different substituents optionally existing on their respective benzene rings, and are each independently selected from the following groups: hydrogen, halogen, C₁₋₆ alkyl, hydroxyl, C₁₋₆ haloalkyl, C₁₋₆ alkoxy, amino, amino mono- or di-substituted with C₁₋₆ alkyl, carboxyl, acetate, amide, and phenyl.

3. The dibenzodiazepine compound according to claim 2, wherein it is a compound of formula II, wherein R¹ is optionally 1-3 different substituents, and is each independently selected from the following groups: hydrogen, halogen, C₁₋₆ alkyl, hydroxyl, and C₁₋₆ alkoxy.

4. The dibenzodiazepine compound according to claim 2, wherein it is a compound of formula II, wherein R² is optionally 1-3 different substituents, and is each independently selected from the following groups: hydrogen, and C₁₋₆ alkoxy.

5. The dibenzodiazepine compound according to claim 1, wherein the dibenzodiazepine compound is selected from the following compounds:
5,11-dihydro-10H-dibenzo[b,f]aza-10-one,
or 6,7-dimethyl-5,11-dihydro-10H-dibenzo[b,f]aza-10-one,
or 6,7-dimethyl-5,11-dihydro-10H-dibenzo[b,f]aza-10-ol,
or 7,8-dimethyl-5,11-dihydro-10H-dibenzo[b,f]aza-10-one,
or 6-chloro-5,11-dihydro-10H-dibenzo[b,f]aza-10-one,
or 7-chloro-5,11-dihydro-10H-dibenzo[b,f]aza-10-one,
or 8-methoxy-5,11-dihydro-10H-dibenzo[b,f]aza-10-one,
or 3-methoxy-6,7-dimethyl-5,11-dihydro-10H-dibenzo[b,f]aza-10-one,
or 2-methoxy-6,7-dimethyl-5,11-dihydro-10H-dibenzo[b,f]aza-10-one,
or 3-fluoro-6,7-dimethyl-5,11-dihydro-10H-dibenzo[b,f]aza-10-one,
or 8-hydroxy-5,11-dihydro-10H-dibenzo[b,f]aza-10-one.

6. The dibenzodiazepine compound according to claim 2, wherein the dibenzodiazepine compound is selected from the following compounds:
5,11-dihydro-10H-dibenzo[b,f]aza-10-one,
or 6,7-dimethyl-5,11-dihydro-10H-dibenzo[b,f]aza-10-one,
or 6,7-dimethyl-5,11-dihydro-10H-dibenzo[b,f]aza-10-ol,
or 7,8-dimethyl-5,11-dihydro-10H-dibenzo[b,f]aza-10-one,
or 6-chloro-5,11-dihydro-10H-dibenzo[b,f]aza-10-one,
or 7-chloro-5,11-dihydro-10H-dibenzo[b,f]aza-10-one,
or 8-methoxy-5,11-dihydro-10H-dibenzo[b,f]aza-10-one,
or 3-methoxy-6,7-dimethyl-5,11-dihydro-10H-dibenzo[b,f]aza-10-one,
or 2-methoxy-6,7-dimethyl-5,11-dihydro-10H-dibenzo[b,f]aza-10-one,
or 3-fluoro-6,7-dimethyl-5,11-dihydro-10H-dibenzo[b,f]aza-10-one,
or 8-hydroxy-5,11-dihydro-10H-dibenzo[b,f]aza-10-one.

7. The dibenzodiazepine compound according to claim 3, wherein the dibenzodiazepine compound is selected from the following compounds:
5,11-dihydro-10H-dibenzo[b,f]aza-10-one,
or 6,7-dimethyl-5,11-dihydro-10H-dibenzo[b,f]aza-10-one,
or 6,7-dimethyl-5,11-dihydro-10H-dibenzo[b,f]aza-10-ol,
or 7,8-dimethyl-5,11-dihydro-10H-dibenzo[b,f]aza-10-one,
or 6-chloro-5,11-dihydro-10H-dibenzo[b,f]aza-10-one,
or 7-chloro-5,11-dihydro-10H-dibenzo[b,f]aza-10-one,
or 8-methoxy-5,11-dihydro-10H-dibenzo[b,f]aza-10-one,
or 3-methoxy-6,7-dimethyl-5,11-dihydro-10H-dibenzo[b,f]aza-10-one,
or 2-methoxy-6,7-dimethyl-5,11-dihydro-10H-dibenzo[b,f]aza-10-one,
or 3-fluoro-6,7-dimethyl-5,11-dihydro-10H-dibenzo[b,f]aza-10-one,
or 8-hydroxy-5,11-dihydro-10H-dibenzo[b,f]aza-10-one.

8. The dibenzodiazepine compound according to claim 4, wherein the dibenzodiazepine compound is selected from the following compounds:
5,11-dihydro-10H-dibenzo[b,f]aza-10-one,
or 6,7-dimethyl-5,11-dihydro-10H-dibenzo[b,f]aza-10-one,
or 6,7-dimethyl-5,11-dihydro-10H-dibenzo[b,f]aza-10-ol,
or 7,8-dimethyl-5,11-dihydro-10H-dibenzo[b,f]aza-10-one,
or 6-chloro-5,11-dihydro-10H-dibenzo[b,f]aza-10-one,
or 7-chloro-5,11-dihydro-10H-dibenzo[b,f]aza-10-one,
or 8-methoxy-5,11-dihydro-10H-dibenzo[b,f]aza-10-one,
or 3-methoxy-6,7-dimethyl-5,11-dihydro-10H-dibenzo[b,f]aza-10-one,
or 2-methoxy-6,7-dimethyl-5,11-dihydro-10H-dibenzo[b,f]aza-10-one,
or 3-fluoro-6,7-dimethyl-5,11-dihydro-10H-dibenzo[b,f]aza-10-one,
or 8-hydroxy-5,11-dihydro-10H-dibenzo[b,f]aza-10-one.

9. The dibenzodiazepine compound according to claim 1, wherein a synthesis intermediate of the dibenzodiazepine compound is a compound of formula III: 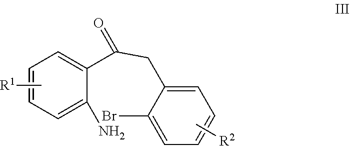
wherein, R¹ and R² are respectively 0-5 different substituents optionally existing on their respective benzene rings, and are each independently selected from the following groups: hydrogen, halogen, C₁₋₆ alkyl, hydroxyl, C₁₋₆ haloalkyl, C₁₋₆ alkoxy, amino, amino mono- or di-substituted with C₁₋₆ alkyl, carboxyl, acetate, amide, and phenyl.

10. The dibenzodiazepine compound according to any one of claim 2, wherein a synthesis intermediate of the dibenzodiazepine compound is a compound of formula III: 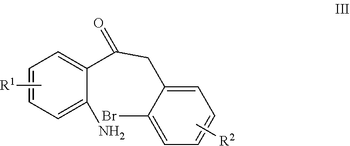
wherein, R1 and R2 are respectively 0-5 different substituents optionally existing on their respective benzene rings, and are each independently selected from the following groups: hydrogen, halogen, C₁₋₆ alkyl, hydroxyl, C₁₋₆ haloalkyl, C₁₋₆ alkoxy, amino, amino mono- or di-substituted with C₁₋₆ alkyl, carboxyl, acetate, amide, and phenyl.

11. The benzodiazepine compound according to claim 9, wherein the compound of formula III is obtained by reacting substituted aniline and substituted o-bromobenzonitrile as raw materials in the presence of boron trichloride and aluminum trichloride, with the reaction pathway as follows:

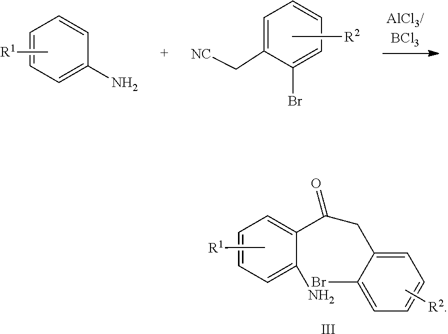

12. The benzodiazepine compound according to claim 10, wherein the compound of formula III is obtained by reacting substituted aniline and substituted o-bromobenzonitrile as raw materials in the presence of boron trichloride and aluminum trichloride, with the reaction pathway as follows:

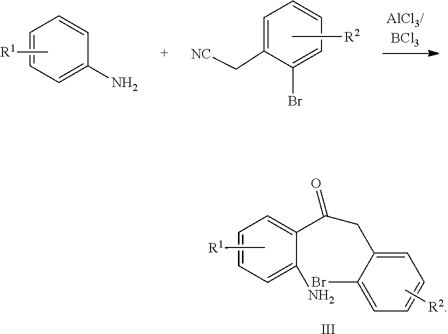

13. Use of the benzodiazepine compound according to claim 2 or a stereoisomer thereof, or a pharmaceutically acceptable salt or solvate thereof in the preparation of drugs for the treatment or prevention of tumors related to the interferon activated protein pathway, as well as diseases caused by viruses and bacteria.
